(19) United States
(12) Patent Application Publication
(10) Pub. No.: US 2002/0161349 A1
(43) Pub. Date: Oct. 31, 2002

(54) Cerebral temperature control

(75) Inventors: Mats Allers, Lund (SE); Anders Lunderquist, Lund (SE); Fredrik Boris-Moller, Lund (SE); Tadeusz Wieloch, Lund (SE)

Correspondence Address: DARBY & DARBY P.C., 805 Third Avenue, New York, NY, 10022, US

(73) Assignee: ARGMED KB, Bjarred (SE)

(21) Appl. No.: 10/072,857

(22) Filed: Feb. 5, 2002

Related U.S. Application Data
(63) Continuation-in-part of application No. PCT/SE01/01259, filed on Jun. 5, 2001.

(30) Foreign Application Priority Data
Jun. 5, 2000 (SE) 0002100-6

(51) Int. Cl. A61M 31/00; A61M 37/00; A61F 7/12

(52) U.S. Cl. 604/500; 604/113; 604/6.13

(57) ABSTRACT

The present invention relates to a method and equipment for controlling the temperature of the brain or brain hemisphere. More specifically the present invention refers to a method and equipment for quick and efficient control of the temperature of the brain and for maintaining a low temperature in the brain. The inventive concept can be divided into three phases. Firstly, an emergency whole body temperature control phase providing a quick temperature control at for example a scene of accident. Secondly, a brain-selective temperature control phase providing a more efficient temperature control at for example an emergency room and thirdly, a maintained temperature control phase providing an even more efficient temperature control for a selected period of time.

This is a continuation-in-part of international application serial number PCT/SE01/01259, filed Jun. 5, 2001, the entire disclosure of which is hereby incorporated by reference.

### TECHNICAL FIELD

The present invention relates to a method and equipment for controlling the temperature of a brain of a living being.

### BACKGROUND OF THE INVENTION

In pathological conditions, the body temperature or the temperature of the body parts of a living being influences the healing process and the risk of permanent damage. Cancer cells, for example, are heat sensitive and a local heating of the blood flow around a cancer tumour may for some types of cancer constitute a treatment resulting in restrained tumour growth, or in some cases even in a shrinking of the tumour. In other cases cooling of a body part may be important to reduce adverse secondary symptoms of the pathological condition.

In the case of a stroke, the blood flow in the brain is reduced due to a haemorrhage or the clogging of a blood vessel. This condition is critical and it is important that treatment is initiated at an early stage, to reduce the loss of bodily functions, such as paralysis. It is well known that cooling the brain effectively blocks the development of cellular damage after an episode of ischemia. Cooling of the patient therefore also results in a reduction of the symptoms of neurological deficit. However, there are certain problems associated with the cooling of an entire patient. One is that the cooling takes a considerable amount of time, another that it must be carried out under close control of vital signs or under anaesthesia, and a third that there is a risk of cardiovascular complications.

In the case of a circulatory arrest, the brain can suffer permanent damage if the arrest exceeds a time period of about 5-15 minutes. However, if the temperature of the brain is lowered before or after the arrest the brain damage is diminished.

In the case of brain trauma the brain suffers from open or close head concussion. Hypothermia has been shown to diminish traumatic brain injury in such cases.

There are several methods in the prior art to carry out a more isolated cooling of a single organ or body part. An example of cooling of the brain in a human being is disclosed in the patent document WO 98/23217, relating to a method of cerebral retro-perfusion and retro-infusion, involving the cooling of arterial blood that then is returned to the entire brain. However, this method entails a large and complicated surgical procedure, which delays the inset of an actual treatment.

The U.S. Pat. No. 5,906,588 discloses a method and a device for heart-lung bypass and cooling of a specific body part. This disclosure primarily relates to complicated heart surgery and organ transplantation.

Today there is no safe and simple method disclosed for a rapid induction of brain hypothermia in ischemic disease.

### PURPOSE OF THE INVENTION

A purpose of the present invention is to provide a system and a method for quick and efficient control of the temperature of the brain. An aim of the invention is to provide a system and a method that is simple and initially does not require specialised personnel acquainted with for example radiology or other diagnostic imaging techniques.

Another purpose of the invention is to provide a system and a method for quick and simple temperature control that also supports subsequent diagnostic measures such as magnetic resonance imaging (MRI), i.e. temperature control without inserting metal components into the body of the patient.

Yet another purpose of the invention is to provide a system and a method for maintaining the temperature control of the brain.

### BRIEF DESCRIPTION OF THE INVENTION

According to the invention the purpose indicated above is achieved by in a first phase, cf. FIG. 1a, introducing an infusion catheter for infusion of a temperature controlled infusion solution or perfusate into a vein initiating a quick general body hypothermia. In an optional second phase of the invention a second infusion catheter is introduced into an artery of the living being. The second infusion catheter is configured to provide selective temperature control of the brain and infusion of other important substrates and pharmacological compounds into the brain. This accomplishes a quick temperature change in the brain, involving only a comparatively minor procedure.

When treating for example a case of stroke, the brain and thus also the affected brain hemisphere can be cooled quickly according to the method of the invention, resulting in a reduction of the symptoms of functional loss. Preferably, continued cooling is maintained in order to keep the temperature of the brain lowered for as long as it takes to diagnose, medicate and restore the functions of the ischaemic brain section.

A further embodiment and possibly a third phase of the invention involves cooling or heating of blood withdrawn from a living being before the blood is recycled to the living being. In this embodiment an extra-corporeal circuit or conduit is established. In one embodiment the extra-corporeal circuit is established between a vein, e.g. a vein in the lower part of the body, and an artery, e.g. the arteria carotis communis, sinister or dexter, arteria subclavia, brachiocephalic trunk, or some other artery that supplies blood to the brain, wherein the temperature of the blood is modified outside the body before the blood is returned to the body through the artery.

Another embodiment contains the method to reintroduce heated blood to the venous system in order to avoid whole body hypothermia. This embodiment thus allows for heating a first part of the body at the same time as a second part, for example the brain, is cooled. The system for performing this embodiment preferably comprises two separate flow branches or circuits, one for the cooling and one for the heating.

Yet another embodiment of the invention involves, in addition to cooling or heating the blood, controlling the oxygenation of the brain and the affected brain hemisphere, i.e. the blood is oxygenated or deoxygenated before it is returned to the body.

The invention comprises a system, equipment components and a method in inter alia the following aspects of the invention:

A method for brain hypothermia, said method comprising, in a first phase to enable an early and fast inset of the hypothermia, the steps of:

providing a container with an infusion solution having a first temperature and a venous infusion catheter connected to an outlet of said container, said venous infusion catheter having an infusion solution lumen;

percutaneously inserting a distal end of said venous infusion catheter into a peripheral vein;

cooling the infusion solution to a second temperature lower than said first temperature; and

infusing a first amount of said cold infusion solution into said vein via the infusion solution lumen of said venous infusion catheter shortly after said cooling, to enable the cold infusion solution to cool the blood flowing to the brain while avoiding air bubbles arising in the infusion solution.

Varieties of this method further comprises a second hypothermia phase for brain-selective hypothermia, wherein an arterial infusion catheter is inserted into an artery and a second amount of cold solution is infused into the arterial system, to enable a more efficient temperature regulation of the brain.

The arterial infusion catheter is inserted into a selected peripheral artery, e.g. an arteria radialis or an arteria brachialis. The method may further comprise the step of positioning a distal tip of said arterial infusion catheter in a selected central artery at the vicinity of a branch artery supplying blood to the brain. The selected central artery is e.g. arteria subclavia at the vicinity of arteria carotis, truncus brachiocephalica or ascending aorta. In some cases it may be necessary or improving to apply a pressure from the outside of the extremity with the peripheral artery for decreasing peripheral blood circulation.

Further embodiments comprise the steps of:

percutaneously inserting a temperature sensor in a blood vessel draining blood from the brain;

sensing the temperature in the blood of said blood vessel thus providing an indication of the temperature in the brain;

adjusting the infusion rate dependent on said sensed temperature for achieving a desired temperature in the brain.

In one embodiment the emergency phase may be directly followed by a hypothermia phase for maintained hypothermia, comprising the steps of:

inserting into a blood vessel an extraction catheter for extraction of blood;

inserting an arterial infusion catheter in the vicinity of an artery supplying blood to the brain;

establishing an first extra-corporeal blood circuit for cooled blood between said extraction catheter and said arterial infusion catheter via a pumping means and a temperature regulating device capable of cooling extracted blood;

extracting blood from said blood vessel via said extraction catheter leading a first amount of said extracted blood into said first extra-corporeal blood circuit;

cooling said first amount of said extracted blood;

infusing said cooled extracted blood to said brain supplying artery via said arterial infusion catheter;

maintaining cooling and circulation in said first extra-corporeal blood circuit for a selected period of time.

A further developed embodiment comprises the steps of:

inserting a venous infusion catheter into a vein of the venous system;

establishing a second extra-corporeal blood circuit for heated blood between said extraction catheter and said venous infusion catheter via said pumping means and a heating device capable of heating extracted blood;

leading a second amount of said extracted blood from said blood vessel via said extraction catheter into said second extra-corporeal blood circuit;

heating said second amount of said extracted blood;

infusing said heated second amount of extracted blood to said venous system via said venous infusion catheter;

maintaining heating and circulation in said second extra-corporeal blood circuit for a selected period of time.

For regulating the temperature this method embodiment further comprises the steps of:

percutaneously inserting a temperature sensor in a blood vessel draining blood from the brain;

sensing the temperature in the blood of said blood vessel thus providing an indication of the temperature in the brain;

adjusting the infusion rate of said cooled blood dependent on said sensed temperature for achieving a desired temperature in the brain, or. the steps of:

percutaneously inserting a temperature sensor in a blood vessel draining blood from the brain;

sensing the temperature in the blood of said blood vessel thus providing an indication of the temperature in the brain;

adjusting the temperature of said cooled blood dependent on said sensed temperature for achieving a desired temperature in the brain.

One embodiment comprises a third hypothermia phase for maintained hypothermia or follows the brain-selective hypothermia phase, the third hypothermia phase comprising the steps of:

inserting into a blood vessel an extraction catheter for extraction of blood;

inserting an arterial infusion catheter in the vicinity of an artery supplying blood to the brain;

establishing a first extra-corporeal blood circuit for cooled blood between said extraction catheter and said arterial infusion catheter via a pumping means and a temperature regulating device capable of cooling extracted blood;

extracting blood from said blood vessel via said extraction catheter leading a first amount of said extracted blood into said first extra-corporeal blood circuit;

cooling said first amount of said extracted blood;

infusing said cooled extracted blood to said brain supplying artery via said arterial infusion catheter;

maintaining cooling and circulation in said first extra-corporeal blood circuit for a selected period of time, and possibly also the steps of:

inserting a venous infusion catheter into a vein of the venous system;

establishing a second extra-corporeal blood circuit for heated blood between said extraction catheter and said venous infusion catheter via said pumping means and a heating device capable of heating extracted blood;

leading a second amount of said extracted blood from said blood vessel via said extraction catheter into said second extra-corporeal blood circuit;

heating said second amount of said extracted blood;

infusing said heated second amount of extracted blood to said venous system via said venous infusion catheter;

maintaining heating and circulation in said second extra-corporeal blood circuit for a selected period of time.

In its most basic form an embodiment of the emergency phase method comprises the steps of:

providing a container with a cold infusion solution and an infusion catheter connected to an outlet of said container, said infusion catheter having an infusion solution lumen;

percutaneously inserting a distal end of said infusion catheter into a blood vessel that supplies the brain with blood;

infusing the cold infusion solution into said blood vessel, to enable the cold infusion solution to flow distally to the brain;

or alternatively phrased, infusing a solution having a first predetermined temperature into a blood vessel supplying said brain hemisphere with blood until said brain hemisphere has reached a predetermined temperature or a predetermined maximum amount of solution has been infused.

An embodiment of an equipment for brain hypothermia in a living being, comprises:

a container of infusion solution;

a temperature regulating apparatus for said infusion solution;

a flexible elongated infusion catheter, said catheter having a proximal end being attachable to an outlet of said container, said catheter having a sufficiently small diameter to be percutaneously insertable into a blood vessel feeding the brain with blood.

Other aspects of the invention include:

An equipment for brain hypothermia, said equipment comprising, to enable an early and fast inset of the hypothermia:

a container with an infusion solution having a first temperature and a venous infusion catheter being connectable to an outlet of said container, said venous infusion catheter having an infusion solution lumen;

said venous infusion catheter having a distal end devised to be percutaneously inserted into a peripheral vein;

a cooling device being configured for cooling the infusion solution to a second temperature lower than said first temperature;

wherein the cooling device is configured for cooling the infusion solution to a second temperature in the range of 0-10 degrees Celsius;

wherein the cooling device is configured for cooling the infusion solution to a second temperature in the range of 0-4 degrees Celsius;

wherein the infusion catheter is configured to be inserted into a median cubital vein;

wherein the infusion catheter is configured to be inserted into a saphenous vein;

wherein the infusion solution is a hypotonic saline solution;

wherein said first amount of infusion solution is in the range of 1-2 liters;

wherein the infusion solution has a low osmolarity in order to lessen the circulatory volume load of the infusion solution when infused into the patient;

wherein the infusion solution is provided in a container that is air-sealed at steady state at a temperature in the range of 37 degrees Celsius;

the equipment further comprising a container with gas having brain protective properties and equipment for inhaling a controlled fraction of said gas.

An embodiment for equipment configured use in a brain-selective hypothermia phase comprises an arterial infusion catheter configured to be inserted into an artery and a container with a second amount of cold solution configured to be infused into the arterial system, to enable a more efficient temperature regulation of the brain in a second hypothermia phase for brain-selective hypothermia.

In different embodiments this equipment:

the arterial infusion catheter is configured to be inserted into a selected peripheral artery;

wherein the arterial infusion catheter is configured to be inserted into an arteria radialis; or an arteria brachialis;

wherein said arterial infusion catheter further is configured to the positioning of a distal tip of said arterial infusion catheter in a selected central artery at the vicinity of a branch artery supplying blood to the brain, wherein said selected central artery is arteria subclavia at the vicinity of arteria carotis, truncus brachiocephalic or ascending aorta.

Embodiments may further comprise a device for applying a pressure from the outside of the extremity with the peripheral artery for decreasing peripheral blood circulation. Further embodiments further comprises:

a temperature sensor configured to be percutaneously inserted in a blood vessel draining blood from the brain;

and being configured to:

sensing the temperature in the blood of said blood vessel thus providing an indication of the temperature in the brain; and

adjusting the infusion rate dependent on said sensed temperature for achieving a desired temperature in the brain.

A further developed embodiment is followed by a third hypothermia phase for maintained hypothermia, and comprises:

an extraction catheter being configured to be inserted into a blood vessel for extraction of blood;

an arterial infusion catheter being configured to be inserted into the vicinity of an artery supplying blood to the brain;

coupling means for establishing an first extra-corporeal blood circuit for cooled blood between said extraction catheter and said arterial infusion catheter via a pumping means and a temperature regulating device capable of cooling extracted blood;

and being configured to:

extracting blood from said blood vessel via said extraction catheter leading a first amount of said extracted blood into said first extra-corporeal blood circuit;

cooling said first amount of said extracted blood;

infusing said cooled extracted blood to said brain supplying artery via said arterial infusion catheter;

maintaining cooling and circulation in said first extra-corporeal blood circuit for a selected period of time;

and possibly further comprising:

a venous infusion catheter being configured to be inserted into a vein of the venous system;

and further being configured to:

establishing a second extra-corporeal blood circuit for heated blood between said extraction catheter and said venous infusion catheter via said pumping means and a heating device capable of heating extracted blood;

leading a second amount of said extracted blood from said blood vessel via said extraction catheter into said second extra-corporeal blood circuit;

heating said second amount of said extracted blood;

infusing said heated second amount of extracted blood to said venous system via said venous infusion catheter;

maintaining heating and circulation in said second extra-corporeal blood circuit for a selected period of time.

In this embodiment a temperature sensor would be configured to adjusting the infusion rate of said cooled blood dependent on said sensed temperature for achieving a desired temperature in the brain; or to adjusting the temperature of said cooled blood dependent on said sensed temperature for achieving a desired temperature in the brain.

An embodiment of an equipment for brain hypothermia comprises, to enable a brain-selective hypothermia:

a container with an infusion solution having a first temperature and an arterial infusion catheter connectable to an outlet of said container, said arterial infusion catheter having an infusion solution lumen;

a distal end of said arterial infusion catheter being configured to be percutaneously inserted into an artery in the vicinity of a branch artery supplying blood to the brain;

a cooling device configured to cooling the infusion solution to a second temperature lower than said first temperature, to enable the cold infusion solution to cool the blood flowing to the brain while avoiding air bubbles arising in the infusion solution and an efficient temperature regulation of the brain.

An embodiment of equipment for brain hypothermia comprises, to enable a maintained hypothermia:

an extraction catheter configured to be inserted into a blood vessel for extraction of blood;

an arterial infusion catheter configured to be inserted in an artery into the vicinity of an artery supplying blood to the brain;

means for establishing an second extra-corporeal blood circuit for cooled blood between said extraction catheter and said arterial infusion catheter via a pumping means and a cooling device capable of cooling extracted blood;

a venous infusion catheter being configured to be inserted into a vein of the venous system;

means for establishing a first extra-corporeal blood circuit for heated blood between said extraction catheter and said venous infusion catheter via said pumping means and a heating device capable of heating extracted blood;

means for extracting blood from said blood vessel via said extraction catheter into said first and second extra-corporeal blood circuit;

a cooling device for cooling a second amount of said extracted blood;

a heating device for heating a first amount of said extracted blood;

and being configured to:

infusing said cooled second amount of extracted blood to said brain supplying artery via said arterial infusion catheter;

infusing said heated first amount of extracted blood to said venous system via said venous infusion catheter.

A basic embodiment of an equipment for brain hypothermia, comprises:

a container with a cold infusion solution and an infusion catheter connectable to an outlet of said container, said infusion catheter having an infusion solution lumen;

a distal end of said infusion catheter being configured to be percutaneously inserted into a blood vessel that supplies the brain with blood;

and being configured to infusing the cold infusion solution into said blood vessel, to enable the cold infusion solution to flow distally to the brain.

A specifically developed catheter, wherein:

the catheter being configured to assume a curvature at its distal part; comprises

a first lumen having a plurality of openings positioned close to a distal end of the catheter and at the outer arc of the curvature;

a second lumen having an opening at the tip of the distal end of the catheter;

a distal part of the catheter tapering from said plurality of openings to said tip of the catheter.

### DESCRIPTION OF THE DRAWINGS

The present invention will be described in further detail below, with reference to the accompanying drawings, of which

FIG. 1a is a block diagram indicating three phases for regulating the brain temperature according to embodiments of the invention;

FIG. 1b is a block diagram indicating the steps of a method according to one embodiment of the invention;

FIG. 2 is a schematic illustration an example of a first embodiment of the system according to the invention, here also called the emergency whole body control phase;

FIG. 3a is a block diagram indicating the steps of a method according to one embodiment of the invention;

FIG. 3b illustrates schematically an example of how a catheter is arranged in the arteria subclavia dexter, in a second embodiment of the system according to the invention, here also called the brain selective temperature control phase;

FIG. 4 is a block diagram indicating the steps of a method according to one embodiment of the invention;

FIG. 5 shows schematically an example of a double lumen catheter applicable in embodiments of the invention;

FIG. 6a illustrates schematically a first example of a the inventive system configured in an embodiment here called the maintained temperature control phase;

FIG. 6b illustrates schematically a second embodiment of the inventive system configured in an embodiment of the maintained temperature control phase;

FIG. 6c illustrates schematically a third embodiment of the inventive system configured in an embodiment of the maintained temperature control phase;

FIG. 7a, 7b and 7c show schematically embodiments of a temperature regulating device; and

FIG. 8 illustrates schematically an embodiment of an extra-corporeal blood circuit comprising a shunt.

### DETAILED DESCRIPTION OF THE INVENTION

The present invention relates to a method, a system and a set of disposable equipment components for controlling the temperature of the brain of a living being. More specifically the present invention refers to a method, a system, and a set of disposable equipment for accomplishing a simple, quick and efficient control of the temperature of a brain. The method according to the invention for accomplishing control of the temperature of the brain and maintaining the control of the temperature during a predetermined period of time can be divided into three phases or procedures. Firstly, an emergency whole body temperature control phase, secondly, a brain selective temperature control phase and thirdly, a maintained temperature control phase.

Below, the invention will be explained by reference to examples of embodiments thereof, primarily relating to cooling the brain in a case of stroke or before and during or after a circulatory arrest in a human patient. In some embodiments of the invention the control of the temperature is performed in conjunction with another treatment procedure, such as infusion or the injection or inhalation of pharmaceuticals or a gas. In the case of inhalation or injection of a gas, the gas should have brain protective properties. Examples of such gases are different anaesthetic gases such as Isofluran and hydrogen gas. By substances having brain protective properties it is here understood such substances that:

decreases the effects of the occurrence of free radicals, that is a substance scavenging free radicals;

decreases the activation of leukocytes; or

decreases inflammatory reactions.

There is also a brain protective effect in adding gas in order to optimise, for example decreasing, the partial pressure of oxygen.

As mentioned above, in the case of a living being suffering from a stroke or being in a state of resuscitation from circulatory arrest, it is very important to provide a quick cooling of the brain in order to minimise the brain damages due to the reduced or loss of blood supply to the brain. Thus, it is important to provide a method for simple and quick temperature control of the brain that may be performed by nursing or ambulance personnel at for example the scene of an accident or at a hospital. In any case, it is obvious to a person skilled in the art that the invention can be adapted to different uses within the scope of the independent claims.

The Emergency Whole Body Temperature Control Phase

To achieve a quick thermal regulation of the selected brain hemisphere, the perfusion may initially consist of some perfusion solution, such as a saline solution or Ringer's acetate, containing an antioxidant or a drug, followed in a second stage by thermally regulated blood which is returned to the patient. According to this (second) embodiment, a compression cuff, such as a tourniquet or the like, is placed on the arm in question, i.e. the arm into which the device for returning the blood is inserted, in order to suppress peripheral circulation and to increase the pressure in the blood vessel, making the pressure of the returning blood equal to the pressure exerted by the heart on the blood, which in consequence results in a flow velocity of 10-12 ml/s. The flow velocity may optionally be regulated in such a way as to make the blood in the arteria vertebralis dexter and the arteria carotis dexter perfuse with a spill-over to the arcus aortae, the flow along the aorta curvatura major being laminar. This is desirable in order to allow the maximum share of the spill-over to reach the aorta carotis sinister. The above arrangement will prevent flow backwards toward the valvular section.

As soon as the thermal regulation has started, diagnostic work can be initiated, such as magnetic resonance imaging, or some other form of diagnostic examination, without having to abort the thermal regulation as described above. When the periferal blood has reached the desired temperature, for example 32° C., the thermal regulation described is aborted, and the temperature reached can be maintained using conventional cooling blankets or the like.

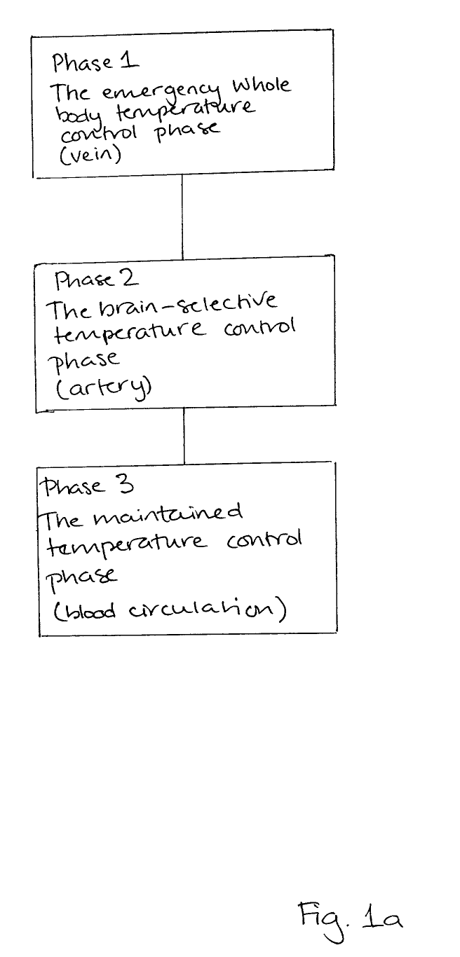

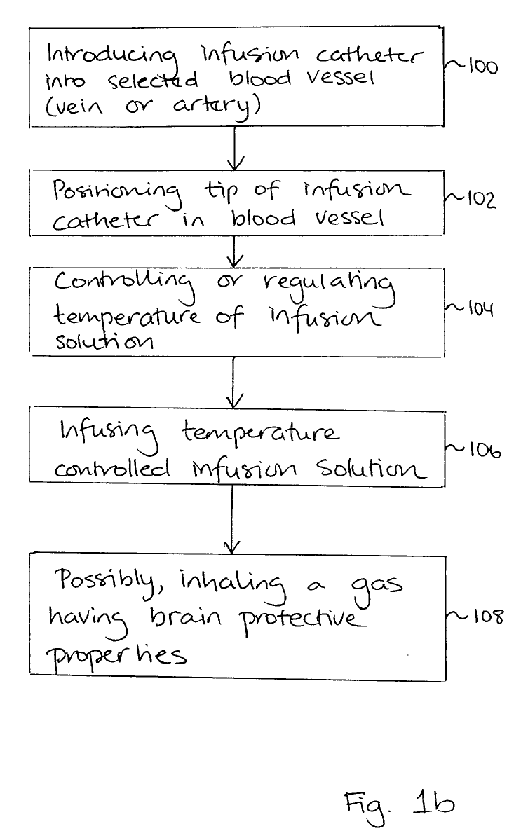

A simple and quick initial control of the temperature of the brain and the body is achieved by the emergency temperature control phase comprising in more general terms the step of initiating as soon as possible an intravenous infusion of a solution that has a controlled temperature, into a blood vessel of the living being by for example ambulance personnel or other nursing staff. In this connection it is possible to add a temperature controlled solution to the body of a human being in the amount of about 1 to 3 liters, or even more. The solution is for example a saline solution possibly comprising antioxidant or other pharmaceuticals having anti-ischaemic properties or suppressing inflammatory processes. These pharmaceuticals may include calcium blocking substances, magnesium or Nimodipin. However, in this description text referring to the whole body temperature control phase, we will refer to the blood vessel as a vein but it could also be an artery if for example personnel that is competent in arterial catheterisation is available. In the case of controlling the temperature during a stroke or a circulatory arrest, the infusion solution is preferably a cold or ice cold solution, e.g., a saline solution possibly comprising an antioxidant. More specifically, embodiments of the emergency temperature control phase comprises as shown in FIG. 1 the steps of:

100 introducing, percutaneously or through a surgical cut down, an infusion catheter into a selected peripheral blood vessel, preferably a vein such as the median cubital vein or saphenous vein, or a central blood vessel such as the jugular vein or the subclavian vein;

102 positioning the tip of the infusion catheter as close to the venous inlet of the heart as possible without getting adverse cardiac effects of the quick infusion of a temperature controlled solution;

104 controlling or regulating the temperature, e.g. cooling, of an infusion solution by means of a temperature regulating means, e.g. a cooling means;

106 infusing the temperature controlled infusion solution, e.g., a cold or ice cold saline solution, preferably having a low osmolarity, into the blood vessel.

One embodiment may also possibly comprise the step of

108 possibly inhaling through a face mask or an endotracheal tube a controlled fraction of gas with brain-protective properties.

Thus, in step 100 an infusion catheter is introduced, preferably percutaneously, into a selected blood vessel, preferably a vein, of the living being such as the median cubital vein, saphenous vein, cephalic vein or basilic vein. For simplicity, the chosen venous catheter is designed such and of the type that the involved personnel are well acquainted with. To achieve sufficient flow of the temperature controlled solution, the inner diameter of the infusion catheter is typically in the range of 1-4 mm, preferably in the range of 2-3 mm, and the length of the infusion catheter is at least about 3 cm. In the case of an infusion catheter long enough to reach for example the intrathoracic space, the tip positioning according to step 102 is preferably performed by means of indication means such as markings arranged on the outer surface of the infusion catheter or small diameter-changes of the catheter, whereby the indication means indicates the length of the catheter that has been introduced into the living being. Thus the position of the catheter tip can be determined without the use of any imaging techniques.

In step 104 the temperature of the infusion solution is regulated or controlled, i.e. the infusion solution is temperature controlled. In the case of stroke or circulatory arrest the infusion solution is preferably a cooled hypotonic saline solution, i.e. a saline solution having an osmotic pressure lower than the blood. An infusion solution having a low osmolarity is preferably chosen in order to lessen the circulatory volume load of the infusion solution. In for example an ambulance, about one to two liters of infusion solution having a temperature in the range of 0-10 degrees Celsius, preferably in the range of 0-4 degrees Celsius, can be infused, whereby a lowered body and brain temperature is achieved. However, in some situations it is sufficient to infuse an infusion solution having a temperature in the range of 10-37 degrees Celsius, since infusion of a infusion solution having a temperature lower than the body temperature will cause a decreased brain temperature.

In one embodiment of the invention, the infusion solution is cooled by means of a peltier element, i.e. an apparatus which by means of electricity, produces cooling of a metal part in an electrical circuit which metal part cools the infusion solution. However, another suitable cooling means can also be used. For example, a container comprising the infusion solution can be placed in an icebox or in a container comprising ice or ice cubes.

However, a cooled infusion solution in steady state with the atmospheric pressure may give rise to gas bubbles or air bubbles of different sizes when supplied to a patient, due to the lower gas solubility at higher temperatures. Such air bubbles may be very harmful to the patient if they are conveyed to the brain. Thus it is important to provide a quick cooling of the solution just before the supply of the solution in order to avoid or decrease the amount of gas or air bubbles in the infusion solution. Another way of avoiding or decreasing the amount of air bubbles is to provide the infusion solution in a sealed or air-sealed container, for example in a sealed container manufactured of a gas impermeable plastic or a plastic-like material, and sealing performed at steady state at a temperature in the range of 37 degrees Celsius. In some cases an amount of small air bubbles can be tolerated.

Further, in step 106 the infusion solution is infused to venous blood by means of the infusion catheter, whereby a lowered body temperature is achieved. In one embodiment of the invention, the body temperature is decreased by one to two degrees Celsius when one to two liters of cooled infusion solution is infused. The infusion speed or velocity can be varied for example by applying a pressure on the container or plastic bag comprising the infusion solution, but the infusion speed should preferably be as large as possible. However, the infusion speed depends on the dimensions of the infusion catheter.

Possibly, in step 108 a gas having brain protective properties is inhaled through for example a facial mask. Examples of such gases are different anaesthetic gases such as NO, Isofluran and hydrogen gas. By substances having brain protective properties it is here understood such substances that decreases the effects of the occurrence of free radicals, that is a substance scavenging free radicals. Further the substances having brain protective properties can decrease the activation of leukocytes or decrease inflammatory reactions. There is also a brain protective effect in adding gas in order to optimise, for example decreasing, the partial pressure of oxygen.

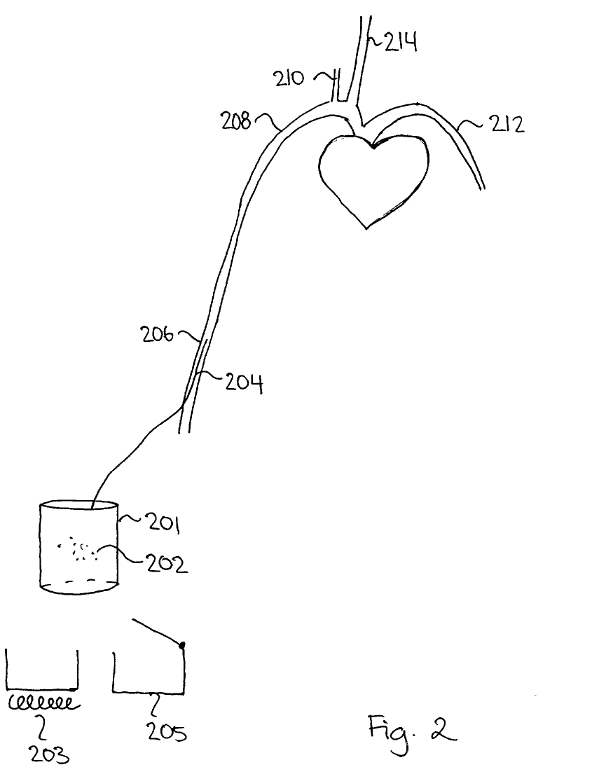

FIG. 2 shows this embodiment of the invention applied in a human patient for cooling the brain. A container 201, for example a plastic bag, having a temperature controlled infusion solution 202, is coupled to an infusion catheter 204. The infusion solution is cooled in a cooler 203 before use or is maintained in a cooling box 205. In this exemplifying figure, the infusion catheter is percutaneously introduced into the right median vein 206 and inserted as close to the venous inlet of the heart as possible without getting adverse cardiac effects due to a quick infusion of a cooled infusion solution. The closer the outlet of the infusion catheter 204 is to the venous inlet of the heart the smaller the temperature loss in the cooled infusion solution before reaching the brain. As the emergency temperature control goes on by infusing in this case cooled solution, the temperature of the whole body is decreased.

In the case of heating a cold patient, the treatment would be analogue but of course with a heated infusion solution.

The Brain-Selective Temperature Control Phase

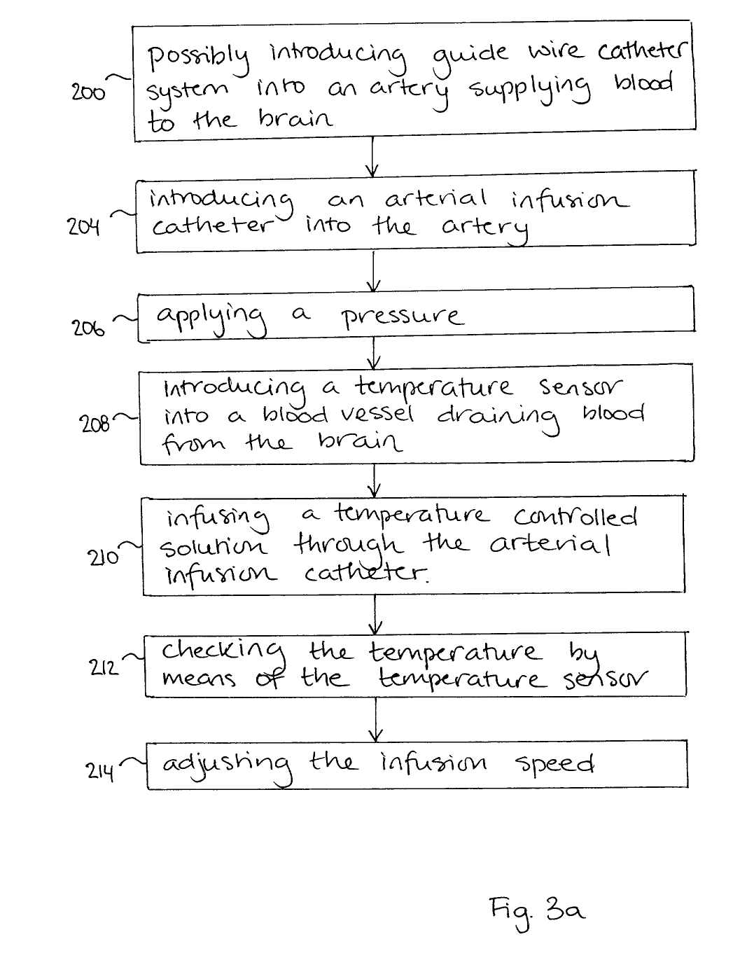

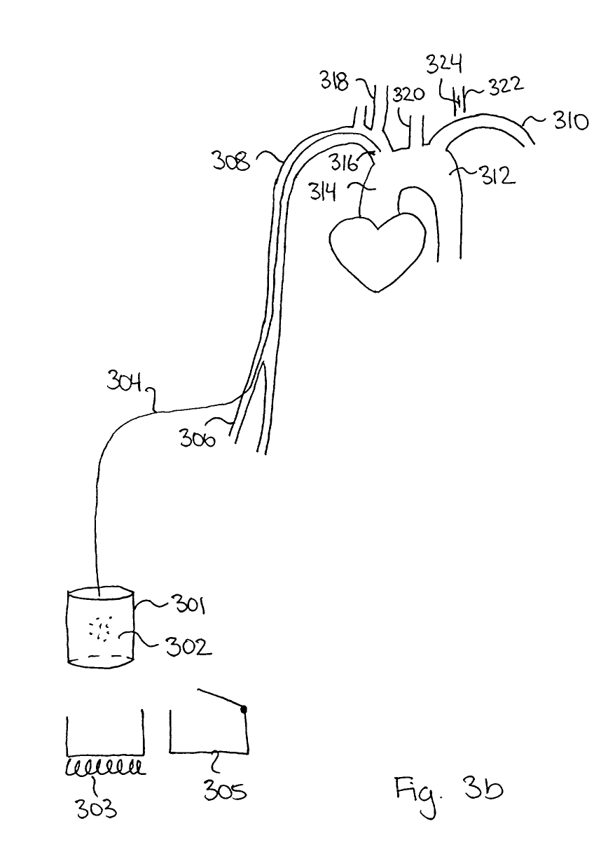

When personnel qualified for arterial catheterisation reaches the patient, for example at a hospital, the brain-selective temperature control phase may be achieved, either as a complement to the emergency whole body temperature control procedure or as separate temperature control procedure. As schematically shown in FIG. 3a, the brain-selective temperature control phase comprises the steps of:

200 possibly introducing by surgical cut-down or percutaneously with e.g. Seldinger technique a guide wire-catheter system into a selected relatively peripheral artery, for example arteria radialis or arteria brachialis to provide a high amount of cooled infusion to the brain;

202 introducing an arterial infusion catheter into the selected artery, for example arteria radialis or arteria brachialis by means of a guide-wire;

204 positioning the tip of the arterial infusion catheter in a selected relatively central or brain close artery, preferably in the right arteria subclavia, truncus brachiocephalica, the ascending aorta, arteria carotis communis or in another selected artery;

206 applying a pressure from the outside on the extremity with the peripheral artery, e.g. the arm or the leg, for example by means of a circumferential torniquet, preventing or decreasing peripheral circulation of the blood, when the cooled solution is infused through the catheter;

208 preferably introducing percutaneously a temperature sensor, such as a thermistor or a thermocouple, into a blood vessel that drains blood from the brain such as the right and/or the left jugular veins. The temperature sensor is preferably coupled to an externally arranged display means displaying the measured temperature of the blood in the brain draining blood vessel, e.g. the jugular vein(s);

210 infusing a temperature controlled solution, e.g., a cold or ice cold saline solution, preferably having a low osmolarity, through the arterial infusion catheter, by means of a supplying means such as a pumping means, e.g., a perfusion pump, to the selected site of infusion;

212 checking the temperature of the brain or the affected brain hemisphere by means of the temperature sensor sensing the temperature in the blood flow in the respective brain draining blood vessels, e.g. the respective jugular vein, thus being dependent on the brain temperature;

214 adjusting the infusion speed or the infusion rate, i.e. the amount of the temperature controlled infusion solution supplied per unit of time, so that a desired and predetermined temperature is achieved in the brain or the affected brain hemisphere, thus preferably dependent on the sensed temperature.

Thus in step 200, a guide wire-catheter system is possibly introduced, percutaneously or by surgical cut-down, into a selected blood vessel supplying blood to the brain. Preferably, the catheter is introduced into the right arteria radialis or the right arteria brachialis, and advanced to the predetermined position, whereby a high concentration of cooled blood can be supplied to the brain. In step 202, a device for infusion of an infusion fluid, preferably consisting of a guide wire and a heparinised and hydrophilic arterial infusion catheter is inserted. The tip of the arterial infusion catheter is in step 204 positioned in for example the right arteria subclavia, arteria brachiocephalica or in the ascending aorta.

A pressure is possibly applied in step 206 on the arm used for the arterial infusion catheter. The pressure is preferably achieved by means of a pressure means, for example by applying a circumferential tourniquet around the selected arm. The pressure means can further be arranged for cooling the arm or for preventing peripheral circulation of blood.

In step 208 a temperature sensor, such as a thermistor or a thermocouple, is preferably percutaneously introduced into for example the right and/or left jugular veins. The temperature sensor is preferably coupled to an externally arranged display means displaying the measured temperature of the blood in the jugular vein(s) to an operator.

In step 210 a temperature controlled infusion solution, e.g. a cold or icecold saline solution, is infused into the selected artery by means of the arterial infusion catheter and a supplying means, such as a pumping means, e.g. a perfusion pump.

Further in step 212, the temperature of the brain or the affected brain hemisphere is controlled by means of the temperature sensor, and in step 214, the infusion speed of the infusion solution is adjusted to achieve a desired and predetermined temperature in the brain or the affected brain hemisphere.

In one embodiment of the invention, the arterial infusion catheter has a diameter in the range of 0.3-3.3 mm (1-10 F, 1 F=0.33 mm), preferably in the range of 1.3-2.6 mm (4-8 F). The surface of the arterial infusion catheter is preferably coated with an anticoagulant compound to prevent coagulation of blood on the catheter surface and in the surroundings of the arterial infusion catheter. Further, in one embodiment of the invention the arterial infusion catheter has an externally arranged indication means, such as markings or diameter-changes, in order to indicate the position of the catheter tip in the selected artery, e.g. the right arteria subclavia, truncus brachiocephalic or ascending aorta. Thus it is possible to guide and position the catheter tip into a selected and predetermined position without using any diagnostic imaging method, such as a radiological method. The dimensions and positioning markings are preferably adapted to the size of the body of the patient.

FIG. 3b shows schematically an example of this embodiment of the invention applied in the arterial system of a human patient. A container 301 containing a temperature controlled infusion solution 302 is connected to an arterial infusion catheter 304. The infusion solution 302 is tempered in a cooler or heater 303 or has a pre-arranged temperature that is maintained by means of a cooling or heating box 305. Most examples in the specification are however directed to cooling.

In this figure, the infusion catheter 304 is inserted into the right radial artery 306 with its distal end outlet introduced up the right subclavian artery 308 in a position at level with the inlet of the right carotid artery 318. In this embodiment, a low infusion flow rate will cause the infusion solution to flow up into the right common carotid 318. If the infusion rate is increased a part of the infusion solution will flow up into the right common carotid 318 and another part of the infusion solution will flow down into the ascending aorta 314 and up into the left common carotid 320. However, if the infusion rate is increased even more, the infusion solution will flow down into the coronary arteries.

In another embodiment of the invention, the infusion catheter 304 is inserted via the right arteria subclavia 308 and the aortic arch 312 into the truncus brachiocepahlica 316. In the embodiment, a low infusion rate will cause the infusion solution to flow up into the right common carotid 318. If the infusion rate is increased a part of the infusion solution will flow up into the right common carotid 318 and another part will flow into the ascending aorta 314 and up into the left common carotid 320. However, if the infusion solution is increased further, the infusion solution will flow down into the coronary arteries.

In yet another embodiment of the invention, the infusion catheter 304 is introduced via for example the left arm and the left arteria subclavia 310 into the ascending aorta 314. In this embodiment, the infusion solution will flow up into the left and right common carotids, 320 and 318, respectively.

In a further embodiment of the invention, the infusion catheter 304 is introduced into the ascending aorta 314 via arteria femoralis and the aortic arch 312, whereby the supplied infusion solution flows up in the left and right common carotid, 320 and 318, respectively. This infusion catheter can for example be a double lumen catheter as described below.

Further, a temperature sensor 324 is introduced into a jugular vein 322, whereby the temperature of the blood from the brain can be monitored and thus a measure of the temperature in the brain is achieved.

In an embodiment of the invention, the infusion catheter 304 is coupled to a perfusion apparatus that generates a pulsating flow of infusion solution. This pulsating flow is then synchronised to the heart cycle such that the cool infusion solution flows up in the left and right carotids, 320 and 318, respectively, when the heart is in systole, i.e. when the heart is contracting and pumping blood to the arteries.

In an embodiment of the invention, the infused solution has a temperature in the interval of 0-20 degrees Celsius, preferably in the interval of 0-10 degrees Celsius, and most preferably in the interval of 0-4 degrees Celsius. Preferably, the infusion solution is held or stored in room temperature or preferably at 37 degrees Celsius and is quickly cooled just before the start of the infusion procedure to decrease or avoid the risk of formation of gas or air bubbles in the blood vessel when the infusion solution is infused. In one embodiment of the invention, the infusion has a low osmolarity in order to lessen the circulatory volume load of the infusion solution.

After the insertion of the arterial infusion catheter, one to two liters of infusion solution having a temperature in the range of 0-4 degrees Celsius can be infused intra-arterially without adverse circulatory effects, whereby a selectively lowered brain temperature is achieved as compared to the whole body temperature control procedure. Further, in some cases a brain temperature in the interval of 15 to 37 degrees Celsius is desirable, but in other cases a brain temperature in the range of 30-37 degrees Celsius is desirable. Preferably, a brain temperature in the range of 27 to 35 degrees Celsius is desired and more preferably a brain temperature of approximately 33 degrees Celsius.

According to an embodiment of the invention, a temperature about 30-35 degrees Celsius in the brain or the affected brain hemisphere can be achieved after about 2-8 minutes, by supplying a cooled infusion solution of a temperature in the range of 0-4 degrees Celsius at a speed in the range of 300-700 milliliters per minute. The achieved temperature, time and flow rate are probably dependent on body size of the patient as well as on the infusion rate.

The flow velocity of the infusion solution can optionally be regulated in a way making the blood in the arteria vertebralis dexter and the arteria carotis dexter to perfuse with a spill-over to the arcus aortae and to flow along the aorta curvatura major into the left carotic artery. This is desirable in order to allow the maximum share of the spill-over to reach the aorta carotis sinister. The above arrangement will prevent flow backwards towards the valvular section.

The Maintained Temperature Control Phase

The maintained temperature control procedure, i.e., the procedure for maintaining the desired level of selected brain hypothermia or hyperthermia, can either be accomplished during or after the whole body temperature control procedure or the brain-selective temperature control procedure described above. Further, the maintained temperature control phase can be achieved by using for example a temperature regulated blanket or mattress or by establishing an extra-corporeal circulation of temperature regulated blood or another temperature regulated solution. This phase is for example accomplished in hospitals wherein immediate access to specialised personnel in cardiology and radiology is provided.

In the inventive concept, this phase comprises a method of establishing an extra-corporeal circulation of temperature controlled blood. The method comprises: withdrawal or extraction of blood from a living being for example suffering of stroke or circulatory arrest, controlling or regulating the temperature of the blood, possibly also regulating the oxygenation of the blood, and reintroducing the blood into the living being. By means of these measures, the temperature of the brain and possibly the rest of the body are controlled. One embodiment of the method comprises the establishment of two extra-corporeal circuits, namely a first circuit for cooling the brain and a second circuit for heating other parts of the body. A variety of this embodiment utilises a peltier element for cooling and the opposite side of the same peltier element for heating.

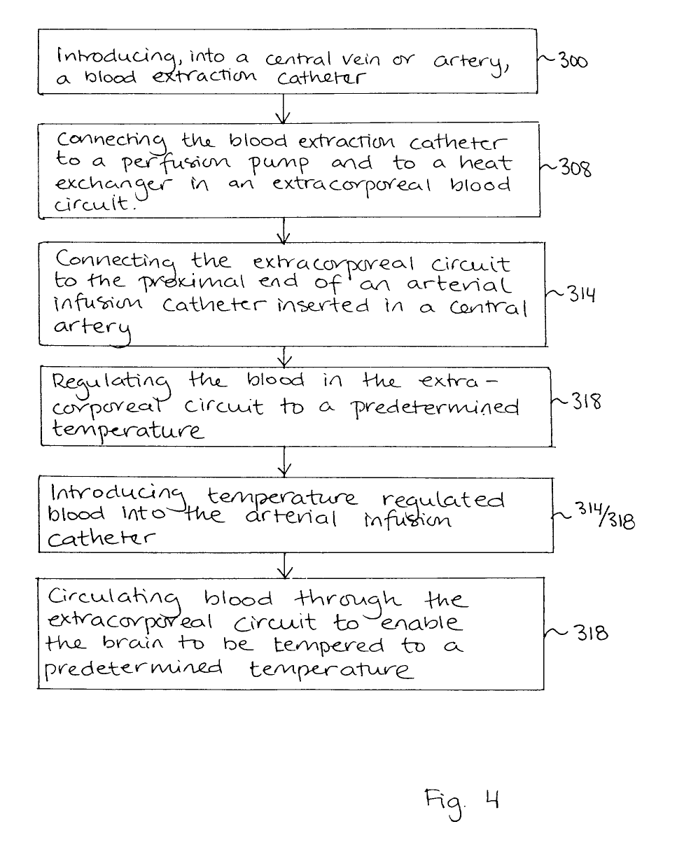

More specifically, an embodiment of the method of establishing an extra-corporeal circulation of temperature controlled blood comprises the steps of (cf. FIG. 4):

300 introducing, into a central vein or artery of a patient, an extraction catheter for extraction of blood;

302 possibly, introducing a venous infusion catheter into a vein of the patient;

304 possibly, introducing an arterial infusion catheter into an artery of the patient, preferably to an artery supplying blood to the brain of the patient;

306 possibly, introducing one or several temperature sensors into one or both jugular veins, whereby the temperature(-s) of the blood in the jugular vein(-s) can be measured;

308 connecting the extraction catheter to an arterial cannula, i.e. the arterial infusion catheter, via a perfusion pump, a heat exchanger and possibly also a regulator regulating the oxygenation of the blood, whereby an extra-corporeal circulation to an artery controlling the temperature of the brain is established;

310 possibly also connecting the extraction catheter to the venous infusion catheter by means of for example a flow-dividing Y-connector, via a second perfusion pump and a heat exchanger, whereby an extra-corporeal circulation to the venous system is established;

312 circulating venous or arterial blood extra-corporeally from the patient through the oxygenator and heat exchanger(-s), regulating or controlling the oxygenation and temperature of the blood, and back to the patient;

314 reintroducing temperature controlled blood into the patient through the arterial infusion catheter arranged in an artery leading blood to the brain, whereby the reintroduced blood regulates the oxygenation and temperature of the brain;

316 optionally, reintroducing temperature controlled blood into the patient through the venous infusion catheter arranged in the vein, whereby the reintroduced blood regulates the temperature of the rest of the body;

318 maintaining the extra-corporeal circulation regulating the temperature at the desired temperature levels of the brain for a desired period of time thereafter;

320 possibly, optimising the viscosity of the blood by infusing a saline solution or by hemofiltration; and

322 applying a heating or cooling blanket in order to maintain a desired body temperature level over an extended period of time.

In step 300, a blood extraction means is inserted into a suitable vein or artery, e.g., vena femoralis or arteria femoralis or vena jugularis. The blood extraction device is in one embodiment of the invention an introducer or a first catheter. For example an extraction catheter having an outer diameter smaller than the normal inner diameter of the vein or the artery, so as not to stop entirely the flow of blood around the extraction catheter, but large enough to give a sufficient flow of extracted blood. The vein or artery is e.g. the vena femoralis or arteria femoralis or vena jugularis. For an adult patient an outer diameter of approximately 2.6-4.6 mm (8-14 F) and an inner diameter as large as possible in the relation to the outer diameter would be sufficient. For children or grown-ups with other vein or artery dimensions, the dimensions of the catheter will obviously have to be modified accordingly. The catheter has in one embodiment of the invention, a conical extra-corporeal coupling for low flow resistance during perfusion. This coupling is provided with a seal that can be perforated, for example by a guide wire and/or a dilator, to prevent unnecessary bleeding. The seal can also be removed for attachment to an extra-corporeal circuit. Preferably, the catheter is heparinised internally and externally, to counteract coagulation of the blood that comes into contact with the catheter.

In step 302, an infusion device is introduced possibly into a sufficiently large peripheral or central vein. The infusion device, is preferably an infusion catheter for the reintroduction or reinfusion of temperature regulated blood or for infusion of a treatment solution. This infusion catheter is primarily intended for the reinfusion of possibly heated extra-corporeally circulated blood, whereby the undesirable cooling effects of the rest of the body may be postponed or completely counteracted.

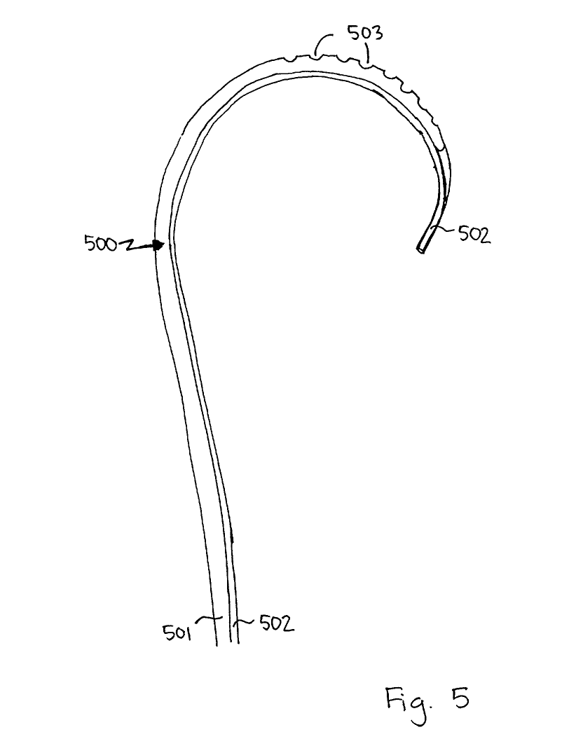

In step 304, an arterial infusion catheter is possibly introduced into an artery supplying blood to the brain. The arterial infusion catheter can be the one used in for example a previous selective brain temperature control phase, whereby it is not necessary to introduce another arterial infusion catheter. However, another arterial infusion catheter can optionally be introduced, for example a double lumen catheter (cf. FIG. 5), which preferably is introduced into the ascending aorta via a femoralis artery. Thus, the optionally introduced arterial infusion catheter can either be used alone or together with another arterial infusion catheter, for example together with an arterial infusion catheter introduced via the left or right arteria subclavia.

In FIG. 5, an exemplifying embodiment of a double lumen catheter 500 is shown. The double lumen catheter has an outer diameter of about 2.7 mm and a first inner lumen 501 having a diameter of about 2.1 mm. In the wall of the catheter is an extra lumen, viz a second inner lumen 502 having a diameter of about 0.3 mm. The distal end of the catheter tapers over a distance of about 3 cm to 1.3 mm still containing the small second inner lumen. The large first inner lumen ends about 4 cm from the tip. The catheter is shaped as a right coronary artery catheter. Extending from the catheter tip and over a distance of approximately 5 cm are a number, for example eight, side holes 503 to the greater lumen of the catheter arranged along the greater curvature of the catheter. The side holes have a diameter of about 1.5 mm.

When the catheter is in position, the tip of the catheter 500 is positioned slightly above the entrance of the left coronary artery and the side holes to the greater lumen are arranged against the right brachiocephalic artery and left carotid artery. By infusion of an infusion solution, such as temperature controlled or regulated blood or a temperature controlled solution at high infusion rate (5-10 ml per second) by means of the larger first inner lumen 501, the infusion solution will flow through the holes 503 against the right brachiocephalic and left carotid artery, whereby rather selective cooling of the brain can be achieved for inducing or maintaining temperature control.

If necessary, the catheter 500 can be advanced some further centimeters, whereby the tip enters the left coronary artery and pharmacological compounds or radiological contrast may be administered locally to the heart by means of the second inner lumen 502.

At least one temperature sensor or a thermistor is in step 306 inserted preferably percutaneously into a jugular vein, whereby the temperature of the blood in the vein can be measured. However, in one embodiment of the invention two temperature sensors are arranged in vena jugularis, sinister and dexter, respectively. By means of the temperature sensors good temperature regulation of the brain is supported. Through the vena jugularis interna, the blood is transported away from the brain, and the thermistor gives off a signal that reflects the temperature of the blood leaving the brain, thus indicating the temperature of the brain or brain hemisphere being thermally regulated. The thermistor preferably consists of some suitable disposable material, which preferably is heparinised. The dimensions of the part inserted into the vena jugularis interna should be small enough to prevent any significant obstruction of the venous blood flow.

The output of the thermistor may be used in different ways. In one embodiment, the signal is transferred to a regulator, which controls the heat exchanger or the circulation pump or both, in order to achieve a regulated temperature level in the selected brain hemisphere. Alternatively, or in addition, the signal may be transferred to an indicating device, such as a visual display showing the current temperature of the blood in the vena jugularis interna, and hence the approximate temperature of the brain or the selected brain hemisphere.

In step 308, the extraction catheter is connected to the arterial cannula, i.e. the return device or the arterial infusion catheter, via a perfusion pump, a heat exchanger and possibly also a regulator regulating the oxygenation of the blood, thereby establishing an extra-corporeal circulation circuit for temperature regulated blood.

Thus the inlet of a blood conduit such as a blood tube is attached according to prior art to the other opening of the extraction catheter, and the tube is passed through a perfusion pump. The blood tube preferably consists of an internally heparinised biocompatible plastic material, and has a diameter suited to its purpose. The blood tube passes through a circulation pump according to prior art, a so-called perfusion pump, preferably equipped with rollers exerting a peristaltic effect externally on the tube.

The blood tube extends from the pump to a heat exchanger, which in this particular embodiment is arranged for cooling the blood, but which in another embodiment may be arranged for heating it or it can be arranged for both heating and cooling. In one type of heat exchanger the blood tube passes through a device which supplies or removes heat energy from the blood through the walls of the blood tube. In another type of heat exchanger, the blood tube is attached to a heparinised heat-exchanging bag with blood canals, providing a large surface area for heating/cooling.

In the embodiment intended for the treatment of stroke or circulatory arrest, the heat exchanger should be capable of regulating blood to a temperature between 0 and 37° C. In some cases, a small temperature fall of only a few degrees is desirable, for example a cooling to 34° C. In other cases a larger temperature fall is desirable, such as down to 0-5° C. Within other areas of application, a heating of the blood may be desirable, such as from 37° C. to about 40 C. As the brain or selected brain hemisphere is cooled/heated, the general body temperature also falls/rises, and accordingly the temperature of the extracted blood. The heat exchanger is therefore controlled so as to keep the blood returned to the body after cooling/heating at the desired temperature.

Optionally, the blood conduit may be attached to a per se known oxygenator or a deoxygenator, before or after the heat exchanger, in order to oxygenate or to deoxygenate the blood.

One outlet end of the extra-corporeal blood circuit is attached to the proximal end of the arterial infusion catheter reaching into the right arteria subclavia, truncus brachiocephalicus, ascending aorta or arteria carotis communis, from the heat exchanger or in relevant cases from the oxygenator or deoxygenator, which completes the configuration of the temperature controlling system and makes it ready for use.

In an embodiment of the present invention, the circulation pump is placed in the proximity of the place of extraction of blood, but it can also be placed elsewhere in the extra-corporeal blood circuit, for example immediately before the blood return catheter. In such a case, the rest of the blood conduit should be primed before starting the infusion of the infusion solution.

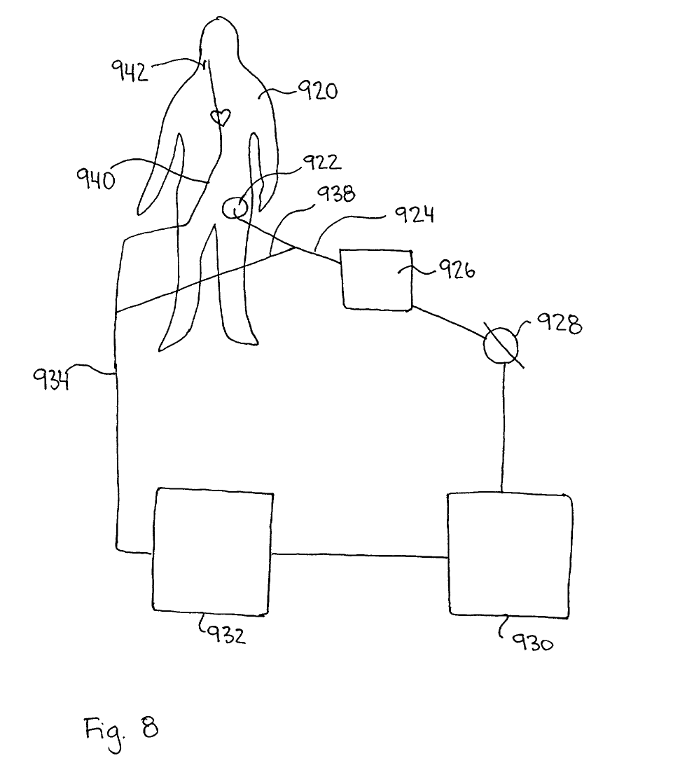

In one embodiment of the invention (cf. FIG. 8), an open reservoir 926 containing, for example, priming solution, such as a saline solution or blood, is arranged between the extraction catheter 922,924 and the circulation pump or the perfusion pump 928, and a shunt 938, in the form of an internally heparinised blood tube, has been arranged extra-corporeally to create a connection from one section between the extraction catheter 922,924 and the reservoir 926 to another section between the artery catheter/infusion catheter 934 and the heat exchanger 932 or the oxygenator/deoxygenator 930. By closing the flow of blood via the extraction catheter 922,924 and opening the flow from the artery, blood will flow out of the arterial catheter 934, and will be pumped by the circulation pump 928 to the reservoir 926, whereby the system will be purged of any air present. Any air present at the extracted blood side, i.e. the venous side or the arterial side, can then be removed similarly by stopping the flow of blood to/from the artery and using the circulation pump 928 to make the extracted blood flow to the reservoir 926. When the system has been purged of air, the blood flow through the shunt 938 is stopped, for example by means of an artery forceps, and the circulation of blood can be started and the temperature of the brain can be monitored by means of a temperature sensor 942 arranged in a jugular vein of the patient 920.

As the brain is continuously selectively cooled, the general body temperature also falls, possibly to undesirable levels. To counteract this effect of brain cooling, optionally a part of the reinfusable blood is in step 310 deviated by way of a Y-formed connector into a heating device and a circulation pump and further introduced into the venous system of the patient. The outlet end of this part of the extra-corporeal blood circuit is attached to the proximal end of the venous infusion catheter.

In step 312, venous or arterial blood is circulated extra-corporeally from the patient through the heat exchanger regulating the temperature of the blood and back to the patient. Thus, when the system for temperature control has been configured, circulation of blood through the extra-corporeal circuit is started, involving the extraction of blood from the vein or artery, cooling or heating it to the desired temperature in the heat exchanger(-s), for example to a temperature between 0 and 37 degrees Celsius, optionally regulating oxygenation by means of an oxygenator, and finally reintroducing it into the patient via the arterial infusion catheter. The temperature controlled blood flows from there into the brain or the affected brain hemisphere, which temperature is controlled swiftly and efficiently.

In step 314, the temperature controlled blood is reintroduced into the patient through the arterial infusion catheter arranged in an artery leading blood to the brain, whereby the reintroduced blood regulates the oxygenation and temperature of the brain or the selected brain hemisphere.

In step 316, heated blood, having a temperature in the range of 37-40 degrees Celsius, is optionally reintroduced into the patient through the venous infusion catheter, whereby undesired general cooling effects on the rest of the body are diminished.

In step 318, the temperature regulated extra-corporeal circulation is maintained at the desired temperature levels of the brain or the affected brain hemisphere for a desired period of time thereafter.

In step 320, the viscosity of the blood can possibly be optimised by infusing a saline solution diluting the blood or by hemofiltration concentrating the blood.

In step 322, external cooling by the means of a cooling blanket may be used for the maintenance of the desired temperature over an extended period of time, also after intra-arterial perfusion has been stopped. Optionally, the blanket may be used for heating the rest of the body during an extended period of intra-arterial perfusion with cooling perfusate, intended for maintenance of local brain hypothermia.

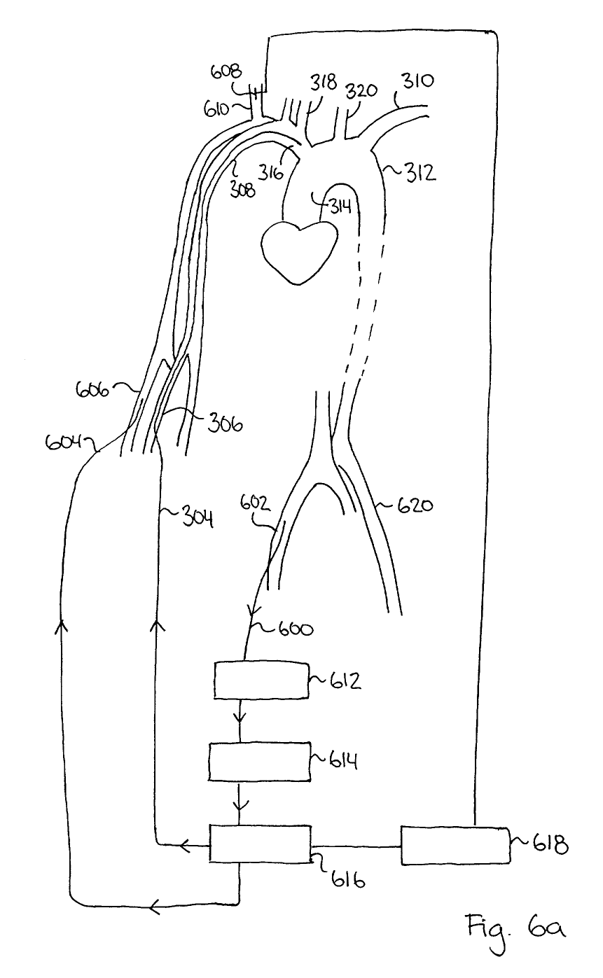

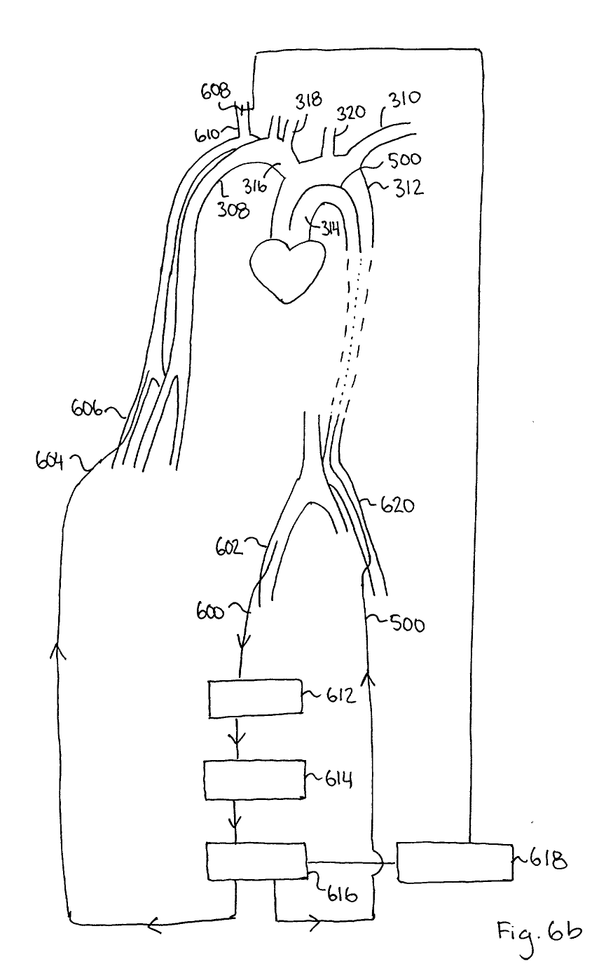

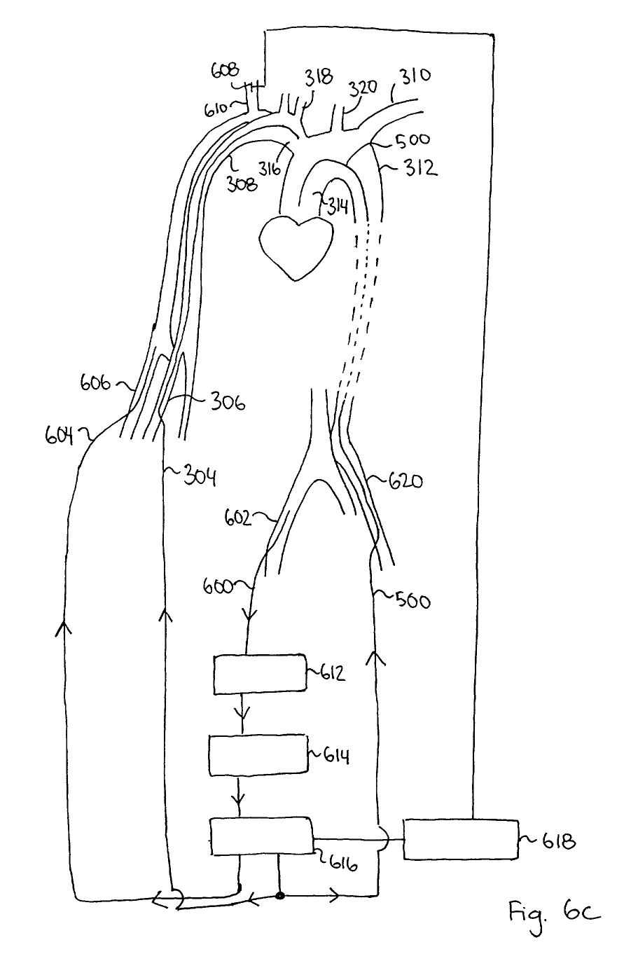

FIG. 6a shows schematically an embodiment of the maintained temperature control phase applied in a human patient. An extraction catheter 600 for extraction of blood is introduced into a central vein or artery of a patient, for example in a femoralis vein 602. Further, a venous catheter 604 is inserted into a vein of the patient, for example the right median vein 606. The extraction catheter 600 is further connected to the venous catheter 604 via a perfusion pump 612, an oxygenator/dexoygenator 614 and a temperature regulator 616, whereby a blood circuit is established supplying heated extracted blood to the patient through the venous catheter 604.

In this embodiment an arterial infusion catheter 306 is introduced into the truncus brachiocephalica 310 through the right radial artery 306 and via the right subclavian artery 308. Further, the arterial infusion catheter is connected to the extraction catheter 600 via the perfusion pump 612, the oxygenator/deoxygenator 614 and the temperature regulator 616, whereby a blood circuit is established supplying cooled extracted blood to the patient via the truncus brachiocephalica 310.

As schematically illustrated in the figure, a temperature sensor 608 is introduced into a jugular vein 610 and possibly connected to a control unit 618 regulating the temperature regulator 616 cooling the extracted blood, whereby the cooling of the brain is controlled or monitored.

In another embodiment of the invention as schematically illustrated in FIG. 6b, an arterial infusion catheter 500 is inserted into the ascending aorta 314 via the left femoral artery 620 and the aortic arch 312. In this embodiment, the cooling blood circuit is established between the extraction catheter 600 and the arterial infusion catheter 500 via the perfusion pump 612, the oxygenator/deoxygenator 614 and the temperature regulator 616. Further, in this embodiment the arterial infusion catheter 500 is the double lumen catheter described with reference to FIG. 5, but it should be understood that the arterial catheter also can be another kind of catheter.

In yet another embodiment of the invention as schematically illustrated in FIG. 6c, three extra-corporeal blood circuits are established. This embodiment of the invention is a combination of the two embodiments previously described with reference to FIGS. 6a and 6b. Firstly, an extra-corporeal blood circuit for heated blood is established between the extraction catheter 600 and the venous infusion catheter 604 via the perfusion apparatus 512, the oxygenator/deoxygenator 614 and the temperature regulating 616. Secondly, an extra-corporeal blood circuit for cooled blood is established between the extraction catheter 600 and the arterial infusion catheter 304 via the perfusion apparatus 512, the oxygenator/deoxygenator 614 and the temperature regulating 616. Thirdly, a third extra-corporeal blood circuit for cooled blood is established between the extraction catheter 600 and the arterial infusion catheter 500 via the perfusion apparatus 512, the oxygenator/deoxygenator 614 and the temperature regulating 616. Thus, in this embodiment having two extra-corporeal blood circuits for cooled blood, a more efficient cooling of the brain is achieved.

Further, in embodiments of the invention the perfusion apparatus 616 is arranged to generate a pulsating flow of infusion solution. This pulsating flow is then synchronised to the heart cycle such that the cooled infusion solution flows up in the left and right carotids, 320 and 318, respectively, when the heart is in systole, i.e. when the heart is contracting and pumping blood to the arteries.

The Temperature Regulating Device

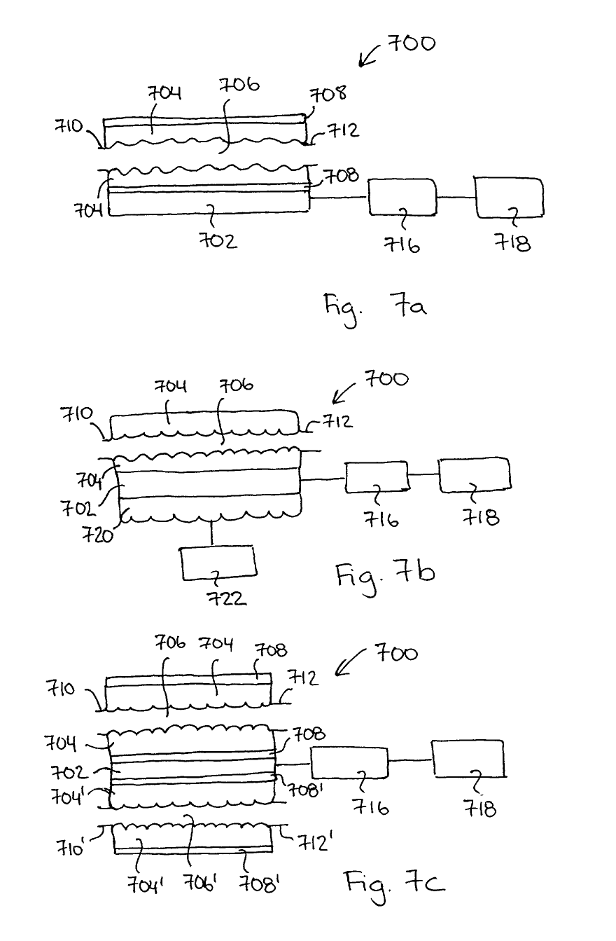

In one embodiment of the invention as schematically illustrated in FIG. 7a, the temperature regulating device or temperature regulator 700 comprises a peltier element 702 and a heat exchanger such as a tube or a conduit 704 of for example folded, twisted or wrinkled stainless steel or another material able to transfer thermal energy. The conduit 704 confines a passage 706 for fluid to be temperature regulated. Thus the conduit 704 has an inlet 710 for fluid to be temperature regulated and an outlet 712 for temperature regulated fluid. If the fluid is to be cooled the conduit 704 should be arranged on the cool side of the peltier element 702 and if the fluid is to be heated the conduit 704 should be arranged on the hot side of the peltier element 702.

In another embodiment of the invention as schematically illustrated in FIG. 7b, a cooling flange 720 is further arranged at the hot side of the peltier element 702, which cooling flange 720 is connected to a fan or a ventilator 722, whereby the hot side of the peltier element 702 is cooled.

In another embodiment of the invention as schematically illustrated in FIG. 7c, the temperature regulating device 700 is used for both cooling and heating a fluid. In this embodiment, the temperature regulating device 700 is designed as a double heat exchanger comprising a peltier element 702 and two conduits 704,704′ confining passages 706,706′ having inlets 710,710′ and outlets 712,712′. In this embodiment, fluid passing though the passages 706,706′ is cooled and heated, respectively.

Further, the temperature regulating device 700 is provided with a power supply 716 supplying power to the peltier element and a control unit 718 controlling the temperature regulation.

In one embodiment of the invention, the conduit 704 or the conduits 704,704′ is/are provided with a plastic or plastic-like housing or cover 708 or covers 708,708′, by means of which the conduit 704 or conduits 704,704′ is/are arranged to be attachable and detachable at the peltier element 702. In this embodiment the conduit 704,704′ and the cover 708,708′ is preferably manufactured as a disposable or disposbales, preferably as a sterile disposable or disposables provided with for example an anticoagulant agent on the inner surface of the conduit 704,704′.

However, in another embodiment of the invention, the temperature regulating device 700 is manufactured as a disposable and preferably as a disposable having sterile inner surfaces of the conduit 704,704′ to not contaminate the fluid to be temperature regulated and inner surfaces provided with an anticoagulant agent.

The present invention has been described above with reference to exemplifying embodiments, and it is obvious to a person skilled in the art that the invention may be modified in other ways within the scope of the appended claims.

1. A method for brain hypothermia, said method comprising, in a first phase to enable an early and fast inset of the hypothermia, the steps of:
providing a container with an infusion solution having a first temperature and a venous infusion catheter connected to an outlet of said container, said venous infusion catheter having an infusion solution lumen;
percutaneously inserting a distal end of said venous infusion catheter into a peripheral vein;
cooling the infusion solution to a second temperature lower than said first temperature; and
infusing a first amount of said cold infusion solution into said vein via the infusion solution lumen of said venous infusion catheter shortly after said cooling, to enable the cold infusion solution to cool the blood flowing to the brain while avoiding air bubbles arising in the infusion solution.

2. The method of claim 1, wherein the infusion solution is cooled to a second temperature in the range of 0-10 degrees Celsius.

3. The method of claim 2, wherein the infusion solution is cooled to a second temperature in the range of 0-4 degrees Celsius.

4. The method of claim 1, wherein the infusion catheter is inserted into a median cubital vein.

5. The method of claim 1, wherein the infusion catheter is inserted into a saphenous vein.

6. The method of claim 1, wherein the infusion solution is a hypotonic saline solution.

7. The method of claim 1, wherein said first amount of infusion solution is in the range of 1-2 liters.

8. The method of claim 1, wherein the infusion solution has a low osmolarity in order to lessen the circulatory volume load of the infusion solution when infused into the patient.

9. The method of claim 1, further comprising the step of providing the infusion solution in a container that is air-sealed at steady state at a temperature in the range of 37 degrees Celsius.

10. The method of claim 1, further comprising the step of inhaling a controlled fraction of gas having brain protective properties.

11. The method of claim 1, further comprising a second hypothermia phase for brain-selective hypothermia, wherein an arterial infusion catheter is inserted into an artery and a second amount of cold solution is infused into the arterial system, to enable a more efficient temperature regulation of the brain.

12. The method of claim 11, wherein the arterial infusion catheter is inserted into a selected peripheral artery.

13. The method of claim 12, wherein the arterial infusion catheter is inserted into an arteria radialis.

14. The method of claim 12, wherein the arterial infusion catheter is inserted into an arteria brachialis.

15. The method of claim 11, further comprising the step of positioning a distal tip of said arterial infusion catheter in a selected central artery at the vicinity of a branch artery supplying blood to the brain.

16. The method of claim 15, wherein said selected central artery is arteria subclavia at the vicinity of arteria carotis.

17. The method of claim 15, wherein said selected central artery is truncus brachiocephalica.

18. The method of claim 15, wherein said selected central artery is ascending aorta.

19. The method of claim 12, further comprising the step of applying a pressure from the outside of the extremity with the peripheral artery for decreasing peripheral blood circulation.

20. The method of claim 11, further comprising the steps of:
percutaneously inserting a temperature sensor in a blood vessel draining blood from the brain;
sensing the temperature in the blood of said blood vessel thus providing an indication of the temperature in the brain;
adjusting the infusion rate dependent on said sensed temperature for achieving a desired temperature in the brain.

21. The method of claim 1, further comprising a third hypothermia phase for maintained hypothermia, comprising the steps of:
inserting into a blood vessel an extraction catheter for extraction of blood;
inserting an arterial infusion catheter in the vicinity of an artery supplying blood to the brain;
establishing an first extra-corporeal blood circuit for cooled blood between said extraction catheter and said arterial infusion catheter via a pumping means and a temperature regulating device capable of cooling extracted blood;
extracting blood from said blood vessel via said extraction catheter leading a first amount of said extracted blood into said first extra-corporeal blood circuit;
cooling said first amount of said extracted blood;
infusing said cooled extracted blood to said brain supplying artery via said arterial infusion catheter;
maintaining cooling and circulation in said first extra-corporeal blood circuit for a selected period of time.

22. The method of claim 21, further comprising the steps of:
inserting a venous infusion catheter into a vein of the venous system;
establishing a second extra-corporeal blood circuit for heated blood between said extraction catheter and said venous infusion catheter via said pumping means and a heating device capable of heating extracted blood;
leading a second amount of said extracted blood from said blood vessel via said extraction catheter into said second extra-corporeal blood circuit;
heating said second amount of said extracted blood;
infusing said heated second amount of extracted blood to said venous system via said venous infusion catheter;
maintaining heating and circulation in said second extra-corporeal blood circuit for a selected period of time.

23. The method of claim 22, further comprising the steps of:
percutaneously inserting a temperature sensor in a blood vessel draining blood from the brain;
sensing the temperature in the blood of said blood vessel thus providing an indication of the temperature in the brain;
adjusting the infusion rate of said cooled blood dependent on said sensed temperature for achieving a desired temperature in the brain.

24. The method of claim 22, further comprising the steps of:
percutaneously inserting a temperature sensor in a blood vessel draining blood from the brain;
sensing the temperature in the blood of said blood vessel thus providing an indication of the temperature in the brain;
adjusting the temperature of said cooled blood dependent on said sensed temperature for achieving a desired temperature in the brain.

25. The method of claim 21, wherein:
said arterial infusion catheter is a double lumen catheter having a first lumen for infusing cooled blood through a plurality of openings at the distal end of said catheter and a second lumen for infusing a substance through an opening at the distal tip of said catheter;
said distal end of said infusion catheter is positioned in ascending aorta such that said plurality of openings of said first lumen are arranged at level with the brachiocephalic artery and the left carotid artery; and
said opening of said distal tip is positioned at the entrance of the coronary artery.

26. The method of claim 11, further comprising a third hypothermia phase for maintained hypothermia, comprising the steps of:
inserting into a blood vessel an extraction catheter for extraction of blood;
inserting an arterial infusion catheter in the vicinity of an artery supplying blood to the brain;
establishing a first extra-corporeal blood circuit for cooled blood between said extraction catheter and said arterial infusion catheter via a pumping means and a temperature regulating device capable of cooling extracted blood;
extracting blood from said blood vessel via said extraction catheter leading a first amount of said extracted blood into said first extra-corporeal blood circuit;
cooling said first amount of said extracted blood;
infusing said cooled extracted blood to said brain supplying artery via said arterial infusion catheter;
maintaining cooling and circulation in said first extra-corporeal blood circuit for a selected period of time.

27. The method of claim 26, further comprising the steps of:
inserting a venous infusion catheter into a vein of the venous system;
establishing a second extra-corporeal blood circuit for heated blood between said extraction catheter and said venous infusion catheter via said pumping means and a heating device capable of heating extracted blood;
leading a second amount of said extracted blood from said blood vessel via said extraction catheter into said second extra-corporeal blood circuit;
heating said second amount of said extracted blood;
infusing said heated second amount of extracted blood to said venous system via said venous infusion catheter;
maintaining heating and circulation in said second extra-corporeal blood circuit for a selected period of time.

28. A method for brain hypothermia comprising, to enable a brain-selective hypothermia, the steps of:
providing a container with an infusion solution having a first temperature and an arterial infusion catheter connected to an outlet of said container, said arterial infusion catheter having an infusion solution lumen;
percutaneously inserting a distal end of said arterial infusion catheter into an artery in the vicinity of a branch artery supplying blood to the brain;
cooling the infusion solution to a second temperature lower than said first temperature; and
infusing an amount of said cold infusion solution into said artery via the infusion solution lumen of said arterial infusion catheter shortly after said cooling, to enable the cold infusion solution to cool the blood flowing to the brain while avoiding air bubbles arising in the infusion solution and an efficient temperature regulation of the brain.

29. A method for brain hypothermia comprising, to enable a maintained hypothermia, the steps of:
inserting into a blood vessel an extraction catheter for extraction of blood;
inserting an arterial infusion catheter in the vicinity of an artery supplying blood to the brain;
establishing an second extra-corporeal blood circuit for cooled blood between said extraction catheter and said arterial infusion catheter via a pumping means and a cooling device capable of cooling extracted blood;
inserting a venous infusion catheter into a vein of the venous system;
establishing a first extra-corporeal blood circuit for heated blood between said extraction catheter and said venous infusion catheter via said pumping means and a heating device capable of heating extracted blood;
extracting blood from said blood vessel via said extraction catheter into said first and second extra-corporeal blood circuit;
cooling a second amount of said extracted blood;
infusing said cooled second amount of extracted blood to said brain supplying artery via said arterial infusion catheter;
heating a first amount of said extracted blood;
infusing said heated first amount of extracted blood to said venous system via said venous infusion catheter.

30. A method for brain hypothermia, said method comprising the steps of:
providing a container with a cold infusion solution and an infusion catheter connected to an outlet of said container, said infusion catheter having an infusion solution lumen;
percutaneously inserting a distal end of said infusion catheter into a blood vessel that supplies the brain with blood;
infusing the cold infusion solution into said blood vessel, to enable the cold infusion solution to flow distally to the brain.

31. A method of achieving thermal regulation of a selected brain hemisphere, said method comprising:
infusing a solution having a first predetermined temperature into a blood vessel supplying said brain hemisphere with blood until said brain hemisphere has reached a predetermined temperature or a predetermined maximum amount of solution has been infused.

32. The method as recited in claim 31, wherein the infusion solution has a predetermined temperature in the range of 0-4 degrees Celsius.

33. The method as recited in claim 31, further comprising the step of:
after the infusion step,
establishing an extra-corporeal blood circuit;
drawing off blood from the patient in said extra-corporeal circuit;
regulating the temperature of said blood to a selectable temperature; and
returning said temperature regulated blood to the patient.

34. An equipment for brain hypothermia in a living being, said equipment comprising:
a container of infusion solution;
a temperature regulating apparatus for said infusion solution;
a flexible elongated infusion catheter, said catheter having a proximal end being attachable to an outlet of said container, said catheter having a sufficiently small diameter to be percutaneously insertable into a blood vessel feeding the brain with blood.

35. An equipment for brain hypothermia, said equipment comprising, to enable an early and fast inset of the hypothermia:
a container with an infusion solution having a first temperature and a venous infusion catheter being connectable to an outlet of said container, said venous infusion catheter having an infusion solution lumen;
said venous infusion catheter having a distal end devised to be percutaneously inserted into a peripheral vein;
a cooling device being configured for cooling the infusion solution to a second temperature lower than said first temperature.

36. The equipment of claim 35, wherein the cooling device is configured for cooling the infusion solution to a second temperature in the range of 0-10 degrees Celsius.

37. The equipment of claim 36, wherein the cooling device is configured for cooling the infusion solution to a second temperature in the range of 0-4 degrees Celsius.

38. The equipment of claim 35, wherein the infusion catheter is configured to be inserted into a median cubital vein.

39. The equipment of claim 35, wherein the infusion catheter is configured to be inserted into a saphenous vein.

40. The equipment of claim 35, wherein the infusion solution is a hypotonic saline solution.

41. The equipment of claim 35, wherein said first amount of infusion solution is in the range of 1-2 liters.

42. The equipment of claim 35, wherein the infusion solution has a low osmolarity in order to lessen the circulatory volume load of the infusion solution when infused into the patient.

43. The equipment of claim 35, wherein the infusion solution is provided in a container that is air-sealed at steady state at a temperature in the range of 37 degrees Celsius.

44. The equipment of claim 35, further comprising a container with gas having brain protective properties and equipment for inhaling a controlled fraction of said gas.

45. The equipment of claim 35, further comprising an arterial infusion catheter configured to be inserted into an artery and a container with a second amount of cold solution configured to be infused into the arterial system, to enable a more efficient temperature regulation of the brain in a second hypothermia phase for brain-selective hypothermia.

46. The equipment of claim 45, wherein the arterial infusion catheter is configured to be inserted into a selected peripheral artery.

47. The equipment of claim 46, wherein the arterial infusion catheter is configured to be inserted into an arteria radialis.

48. The equipment of claim 46, wherein the arterial infusion catheter is configured to be inserted into an arteria brachialis.

49. The equipment of claim 45, wherein said arterial infusion catheter further is configured to the positioning of a distal tip of said arterial infusion catheter in a selected central artery at the vicinity of a branch artery supplying blood to the brain.

50. The equipment of claim 49, wherein said selected central artery is arteria subclavia at the vicinity of arteria carotis.

51. The equipment of claim 49, wherein said selected central artery is truncus brachiocephalica.

52. The equipment of claim 49, wherein said selected central artery is ascending aorta.

53. The equipment of claim 46, further comprising a device for applying a pressure from the outside of the extremity with the peripheral artery for decreasing peripheral blood circulation.

54. The equipment of claim 45, further comprising:
a temperature sensor configured to be percutaneously inserted in a blood vessel draining blood from the brain;
and being configured to:
sensing the temperature in the blood of said blood vessel thus providing an indication of the temperature in the brain; and
adjusting the infusion rate dependent on said sensed temperature for achieving a desired temperature in the brain.

55. The equipment of claim 35, further, for a third hypothermia phase for maintained hypothermia, comprising:
an extraction catheter being configured to be inserted into a blood vessel for extraction of blood;
an arterial infusion catheter being configured to be inserted into the vicinity of an artery supplying blood to the brain;
coupling means for establishing an first extra-corporeal blood circuit for cooled blood between said extraction catheter and said arterial infusion catheter via a pumping means and a temperature regulating device capable of cooling extracted blood;
and being configured to:
extracting blood from said blood vessel via said extraction catheter leading a first amount of said extracted blood into said first extra-corporeal blood circuit;
cooling said first amount of said extracted blood;
infusing said cooled extracted blood to said brain supplying artery via said arterial infusion catheter;
maintaining cooling and circulation in said first extra-corporeal blood circuit for a selected period of time.

56. The equipment of claim 55, further comprising:
a venous infusion catheter being configured to be inserted into a vein of the venous system;
and further being configured to:
establishing a second extra-corporeal blood circuit for heated blood between said extraction catheter and said venous infusion catheter via said pumping means and a heating device capable of heating extracted blood;
leading a second amount of said extracted blood from said blood vessel via said extraction catheter into said second extra-corporeal blood circuit;
heating said second amount of said extracted blood;
infusing said heated second amount of extracted blood to said venous system via said venous infusion catheter;
maintaining heating and circulation in said second extra-corporeal blood circuit for a selected period of time.

57. The equipment of claim 56, further comprising:
a temperature sensor being configured to be percutaneously inserted in a blood vessel draining blood from the brain;
and being configured to:
sensing the temperature in the blood of said blood vessel thus providing an indication of the temperature in the brain;
adjusting the infusion rate of said cooled blood dependent on said sensed temperature for achieving a desired temperature in the brain.

58. The equipment of claim 56, further comprising:
a temperature sensor being configured to be percutaneously inserted in a blood vessel draining blood from the brain;
and being configured to:
sensing the temperature in the blood of said blood vessel thus providing an indication of the temperature in the brain;
adjusting the temperature of said cooled blood dependent on said sensed temperature for achieving a desired temperature in the brain.

59. The equipment of claim 55, wherein:
said arterial infusion catheter is a double lumen catheter having a first lumen for infusing cooled blood through a plurality of openings at the distal end of said catheter and a second lumen for infusing a substance through an opening at the distal tip of said catheter;
said distal end of said infusion catheter is positioned in ascending aorta such that said plurality of openings of said first lumen are arranged at level with the brachiocephalic artery and the left carotid artery; and
said opening of said distal tip is positioned at the entrance of the coronary artery.

60. The equipment of claim 45, further comprising:
an extraction catheter being configured to be inserted into a blood vessel for extraction of blood;
an arterial infusion catheter being configured to be inserted into an artery to the vicinity of a branch artery supplying blood to the brain;
means for establishing a first extra-corporeal blood circuit for cooled blood between said extraction catheter and said arterial infusion catheter via a pumping means and a temperature regulating device capable of cooling extracted blood;
means for extracting blood from said blood vessel via said extraction catheter leading a first amount of said extracted blood into said first extra-corporeal blood circuit;
a cooling device for cooling said first amount of said extracted blood;
and being configured to:
infusing said cooled extracted blood to said brain supplying artery via said arterial infusion catheter;
maintaining cooling and circulation in said first extra-corporeal blood circuit for a selected period of time in a third hypothermia phase for maintained hypothermia,

61. The equipment of claim 60, further comprising:
a venous infusion catheter being configured to be inserted into a vein of the venous system;
and being further configured to:
establishing a second extra-corporeal blood circuit for heated blood between said extraction catheter and said venous infusion catheter via said pumping means and a heating device capable of heating extracted blood;
leading a second amount of said extracted blood from said blood vessel via said extraction catheter into said second extra-corporeal blood circuit;
heating said second amount of said extracted blood;
infusing said heated second amount of extracted blood to said venous system via said venous infusion catheter;
maintaining heating and circulation in said second extra-corporeal blood circuit for a selected period of time.

62. An equipment for brain hypothermia comprising, to enable a brain-selective hypothermia:
a container with an infusion solution having a first temperature and an arterial infusion catheter connectable to an outlet of said container, said arterial infusion catheter having an infusion solution lumen;
a distal end of said arterial infusion catheter being configured to be percutaneously inserted into an artery in the vicinity of a branch artery supplying blood to the brain;
a cooling device configured to cooling the infusion solution to a second temperature lower than said first temperature, to enable the cold infusion solution to cool the blood flowing to the brain while avoiding air bubbles arising in the infusion solution and an efficient temperature regulation of the brain.

63. An equipment for brain hypothermia comprising, to enable a maintained hypothermia:
an extraction catheter configured to be inserted into a blood vessel for extraction of blood;
an arterial infusion catheter configured to be inserted in an artery into the vicinity of an artery supplying blood to the brain;
means for establishing an second extra-corporeal blood circuit for cooled blood between said extraction catheter and said arterial infusion catheter via a pumping means and a cooling device capable of cooling extracted blood;
a venous infusion catheter being configured to be inserted into a vein of the venous system;
means for establishing a first extra-corporeal blood circuit for heated blood between said extraction catheter and said venous infusion catheter via said pumping means and a heating device capable of heating extracted blood;
means for extracting blood from said blood vessel via said extraction catheter into said first and second extra-corporeal blood circuit;
a cooling device for cooling a second amount of said extracted blood;
a heating device for heating a first amount of said extracted blood;
and being configured to:
infusing said cooled second amount of extracted blood to said brain supplying artery via said arterial infusion catheter;
infusing said heated first amount of extracted blood to said venous system via said venous infusion catheter.

64. An equipment for brain hypothermia, comprising:
a container with a cold infusion solution and an infusion catheter connectable to an outlet of said container, said infusion catheter having an infusion solution lumen;
a distal end of said infusion catheter being configured to be percutaneously inserted into a blood vessel that supplies the brain with blood;
and being configured to infusing the cold infusion solution into said blood vessel, to enable the cold infusion solution to flow distally to the brain.

65. A catheter, wherein:
the catheter is configured to assume a curvature at its distal part and having
a first lumen having a plurality of openings positioned close to a distal end of the catheter and at the outer arc of the curvature;
a second lumen having an opening at the tip of the distal end of the catheter;
a distal part of the catheter tapering from said plurality of openings to said tip of the catheter.

66. The catheter of claim 65, wherein said curvature is configured to be positioned in and fit to the shape of the aortic arch, said plurality of openings of said first lumen being positioned before the inlet of right brachiocephalic and the left carotid artery.

67. The catheter of claim 65, having an outer diameter of about 2.7 millimeters, and wherein an inner diameter of said first lumen is about 2.1 millimeters and an inner diameter of said second lumen is about 0.3 millimeters.

68. The catheter of claim 65, wherein said plurality of openings of said first lumen are arranged about 4 centimeters from the tip of the catheter, and said distal part tapers ove-r a length of about 3 centimeters containing the second lumen.
